(19) United States
(12) Patent Application Publication
(10) Pub. No.: US 2015/0127087 A1
(43) Pub. Date: May 7, 2015

(54) METHOD OF MANUFACTURING STENT ATTACHED TO ARTIFICIAL BLOOD VESSEL AND STENT ATTACHED TO ARTIFICIAL BLOOD VESSEL MANUFACTURED BY THE SAME

(71) Applicant: BCM Co., Ltd., Koyang-si (KR)

(72) Inventor: Joon-sang KIM, Incheon-Si (KR)

(21) Appl. No.: 14/530,835

(22) Filed: Nov. 3, 2014

(30) Foreign Application Priority Data
Nov. 6, 2013 (KR) 10-2013-0133887
Jan. 7, 2014 (KR) 10-2014-0001922

(51) Int. Cl. A61F 2/90; B29D 23/00; A61F 2/07

(52) U.S. Cl. CPC A61F 2/90; A61F 2/07; B29D 23/00; A61F 2240/001; A61F 2210/0014; A61F 2002/072
USPC 623/1.13; 156/148

(57) ABSTRACT

A stent may be inserted and placed in a lesion portion that is being stenosed or has been stenosed in a blood vessel or a lumen in the body to expand the lesion portion that is being stenosed or has been stenosed. Further, the stent and the artificial blood vessel layers are integrally formed with each other without floating between the stent and the artificial blood vessel layers. Accordingly, foods or other contents travelling through the pathway of a lumen in the body or blood in the vessel may be prevented from contacting the lesion portion, and the progress of the stenosis of the lesion portion may be prevented that may occur as the lesion portion grows toward the inside of the hollow cylindrical body of the stent.

### CROSS-REFERENCE TO RELATED APPLICATIONS

This patent application claims priority under 35 U.S.C. §119 to Korean Patent Application No. 10-2014-0001922 filed on Jan. 7, 2014 and Korean Patent Application No. 10-2013-0133887 filed on Nov. 6, 2013, in the Korean Intellectual Property Office, the disclosures of which are incorporated by reference herein in their entireties.

### TECHNICAL FIELD

Embodiments of the present invention relate to a method of manufacturing a artificial blood vessel-attached stent and a artificial blood vessel-attached stent manufactured by the same, wherein the stent is inserted and placed in a lesion portion that is being stenosed or has been stenosed in a blood vessel or a lumen, such as the esophagus, duodendum, or colon, in the body to expand the lesion portion that is being stenosed or has been stenosed, wherein inner or outer artificial blood vessel layers of the stent are attached to the stent while surrounding the stent, and the inner or outer artificial blood vessel layers are fused together by heating to be integrally attached with the stent.

### DISCUSSION OF RELATED ART

Typically, a medical stent is used as an expanding device that is inserted and placed in a lesion portion that is being stenosed or has been stenosed in a a blood vessel or a lumen, such as the esophagus, duodendum, or colon, in the body to expand the pathway of the lesion portion.

There are disclosed conventional stents of various structures. For example, Korean Patent Nos. 10-424290, 10-457629, and 10-457630 disclose stents formed by crossing and weaving one or more superelastic shape memory alloy wires to form a hollow cylindrical body, jointly connecting both ends thereof, and performing thermal treatment on the same to allow the same to memory its shape.

Such medical stent has elastic forces that restore the stent to its initial state, i.e., elastic forces in a diameter direction and longitudinal direction of the cylindrical body.

The medical stent undergoes a reduction in the volume of the hollow cylindrical body and is inserted and placed in a lesion portion of a blood vessel or lumen in the body by way of an inserting tool or pusher catheter. The stem outwardly pushes the lesion portion the lesion portion that is being stenosed or has been stenosed to thereby expand the pathway of the blood vessel or lumen in the body.

Further, in the above-described conventional medical stents, as disclosed in Korean Patent Nos. 10-351317, 10-1116052, and 10-189094 and Korean Patent Application No. 10-2010-45342, a cylindrical artificial blood vessel (PTFE; Polytetrafluoroethylene) layer is attached on an outer side of the cylindrical body of the stent, and the same is bound with a wire.

By doing so, foods and other contents travelling through the pathway of a lumen in the body or blood in the vessels may be prevented from contacting the lesion portion, and the lesion portion may be prevented from growing toward the inside of the hollow cylindrical body of the stent. Accordingly, the lesion portion may be prevented from being stenosed to the inside of the stent.

However, the conventional stents having artificial blood vessel layers are produced by simply covering the artificial blood vessel layers on the outer side of the stent and binding the same with a wire. Accordingly, the conventional stents may suffer from the problem that the hollow cylindrical body of the stent may be floated from the artificial blood vessel layers to insufficiently exert the above-listed effects.

### SUMMARY

The present invention has been designed to solve the above problems of the conventional art, and according to the present invention, an inner or outer artificial blood vessel layers of the stent are attached to the stent while surrounding the stent, and the inner or outer artificial blood vessel layers are fused together by heating to be integrally attached with the stent, to form an artificial blood vessel attached layer. Accordingly, the present invention may provide better functionality and efficiency.

The present invention is characterized to provide a method of manufacturing an artificial blood vessel-attached stent, the method comprising: forming a stent having a hollow cylindrical body by crossingly weaving a superelastic shape memory alloy wire; performing attachment preparation by forming an inner artificial blood vessel layer by inclinedly winding an artificial blood vessel (PTFE: Polytetrafluoroethylene) along an outer side of a SUS rod to form an inner artificial blood vessel layer, inserting the inner artificial blood vessel layer into the stent, inclinedly winding an artificial blood vessel (PTFE; Polytetrafluoroethylene) along an outer side of the stent to form an outer artificial blood vessel layer, and inserting a silicone tube; and fixedly mounting the SUS rod inside a vacuum heating device, attaching the inner artificial blood vessel layer and the outer artificial blood vessel layer positioned at the inside and outside of the stent to each other by a vacuuming operation towards the stent with the inner artificial blood vessel layer and the outer artificial blood vessel layer internally and externally wrapping around the stent, and thermally fusing the inner artificial blood vessel layer and the outer artificial blood vessel layer to each other by a heating operation so that the inner artificial blood vessel layer and outer artificial blood vessel layer are integrally attached to the stent.

The present invention is characterized to provide an artificial blood vessel-attached stent comprising an artificial blood vessel layer at an outside of a hollow cylindrical body formed by crossingly weaving a superelastic shape memory alloy wire, wherein the artificial blood vessel layer is formed by thermally fusing an inner artificial blood vessel layer and an outer artificial blood vessel layer respectively positioned at an inside and outside of the hollow cylindrical body of the stent, with the inner artificial blood vessel layer and the outer artificial blood vessel layer attached with each other while internally and externally wrapping around the hollow cylindrical body.

As such, according to the present invention, an inner or outer artificial blood vessel layers of the stent are attached to the stent while surrounding the stent, and the inner or outer artificial blood vessel layers are fused together by heating to be integrally attached with the stent, to form an artificial blood vessel attached layer. The stent may be inserted and placed in a lesion portion that is being stenosed or has been stenosed in a blood vessel or a lumen in the body to expand the lesion portion that is being stenosed or has been stenosed.

Further, the stent and the artificial blood vessel layers are integrally formed with each other without floating between the stent and the artificial blood vessel layers. Accordingly, foods or other contents travelling through the pathway of a lumen in the body or blood in the vessel may be prevented from contacting the lesion portion, and the progress of the stenosis of the lesion portion may be prevented that may occur as the lesion portion grows toward the inside of the hollow cylindrical body of the stent.

In other words, the present invention may provide stent products of enhanced quality.

### BRIEF DESCRIPTION OF DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a front view illustrating an example stent;

FIGS. 2A, 2B, 2C, and 2D are plan views illustrating a process of preparing for attachment by inserting an artificial blood vessel layer, a stent, and a silicone tube in a SUS rod;

FIGS. 3A, 3B, 3C, and 3D, respectively, are side cross-sectional views of FIGS. 2A, 2B, 2C, and 2D;

FIG. 4 is a plan view illustrating an attaching process performed by a vacuum heating device according to the present invention;

FIG. 5 is a front view illustrating a stent attached according to the present invention;

FIG. 6 is a side cross-sectional view of FIG. 5;

FIG. 7 is an expanded cross-sectional view of portion “A” of FIG. 6;

FIG. 8 is a front view illustrating another example stent;

FIGS. 9 to 12 are plan views illustrating a process of preparing of attachment by inserting an artificial blood vessel layer, a stent, and a silicone tube in a SUS rod using the stent shown in FIG. 8;

FIGS. 13 to 16, respectively, are side cross-sectional views of FIGS. 9 to 12;

FIG. 17 is a plan view illustrating a process of attaching the stent of FIG. 8 by a vacuum heating device;

FIG. 18 is a front view illustrating the attached stent of FIG. 8;

FIG. 19 is a side cross-sectional view of FIG. 18;

FIG. 20 is an expanded cross-sectional view of portion “‘B’” of FIG. 19; and

FIG. 21 is a view illustrating an example of performing a procedure on the stomach using the attached stent of FIG. 18.

### DETAILED DESCRIPTION OF EMBODIMENTS

Hereinafter, exemplary embodiments of the inventive concept will be described in detail with reference to the accompanying drawings. The inventive concept, however, may be modified in various different ways, and should not be construed as limited to the embodiments set forth herein. As used herein, the singular forms “a,” “an,” and “the” are intended to include the plural forms as well, unless the context clearly indicates otherwise. It will be understood that when an element or layer is referred to as being “on,” “connected to,” “coupled to,” or “adjacent to” another element or layer, it can be directly on, connected, coupled, or adjacent to the other element or layer, or intervening elements or layers may be present.

A method of manufacturing an artificial blood vessel-attached stent according to the present invention is described in detail with reference to the accompanying drawings.

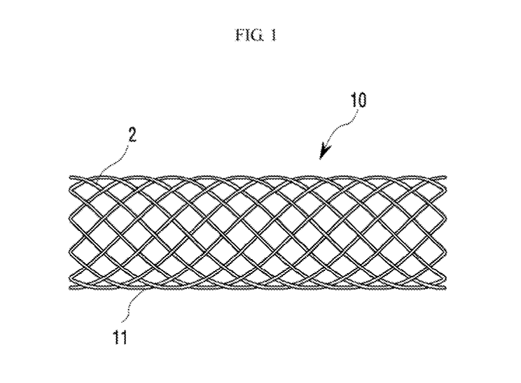

First, as shown in FIG. 1, one or more superelastic shape memory alloy wires 2 are crossed and woven, and both ends thereof are connected with each other, thus forming a stent 10 having a hollow cylindrical body 11.

In this case, the stent 10 is formed by a jig. To form the hollow cylindrical body 11, the jig forms a groove in a longitudinal direction at an equiangular position on the circumference of the cylindrical body, detachably couples pins to the groove at a predetermined distance, hangs the superelastic shape memory allow wires to the pins in a zig-zag pattern along the pins, and weaves the wires while bending and crossing the wires.

In particular, the hollow cylindrical body 11 of the stent may be formed to have various shapes and functions according to the patterns in which the superelastic shape memory alloy wires are hung to the jig pins in a zig-zag shape and woven, bent and crossed. According to the present invention, such stents having various shapes and functions all belong to the scope of the present invention.

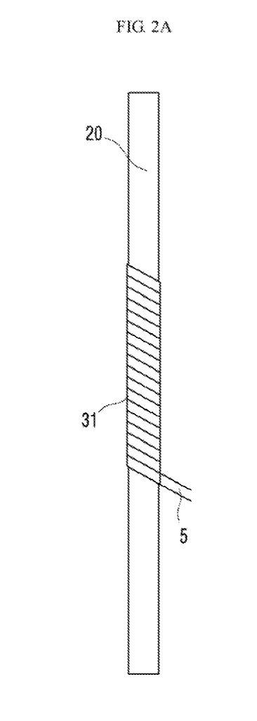

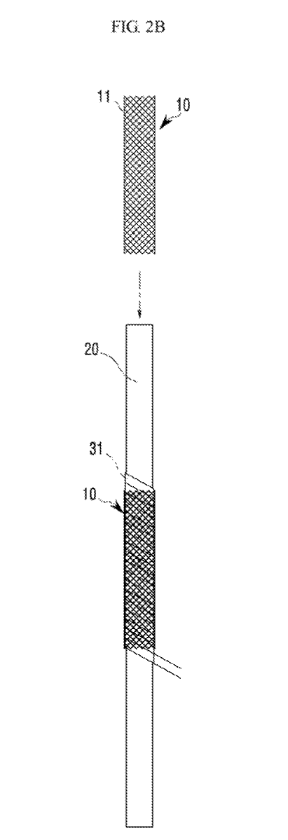

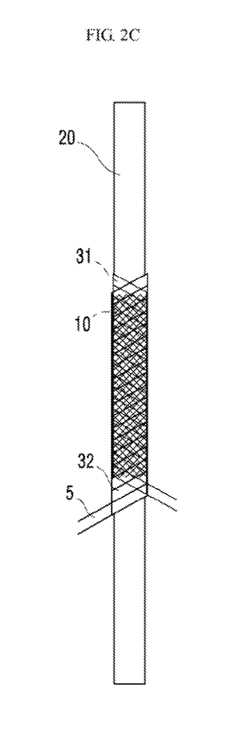

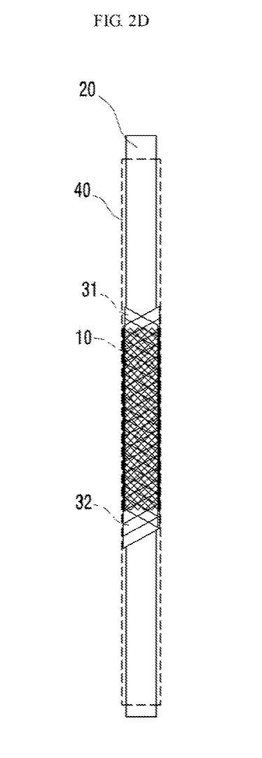

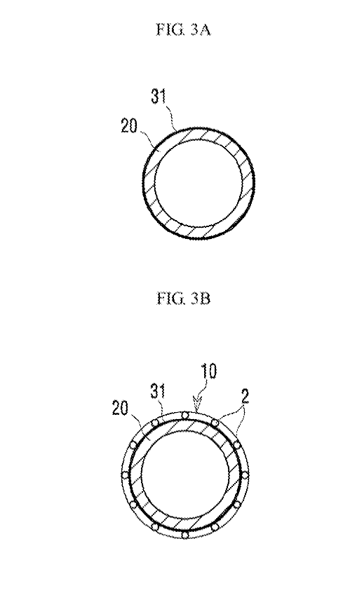

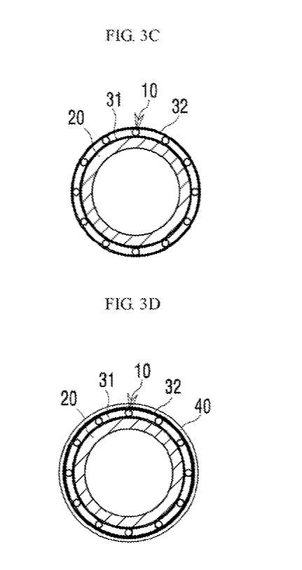

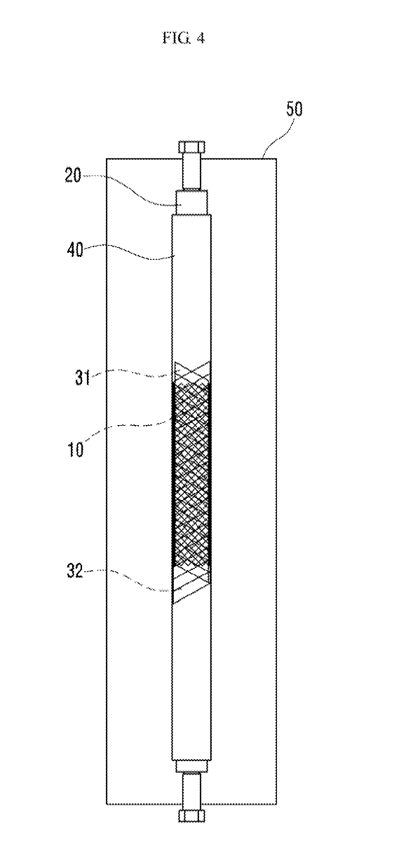

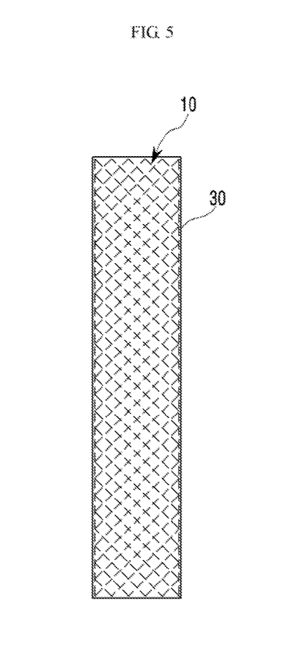

After forming the stent 10, an artificial blood vessel (PTFE; Polytetrafluoroethylene) 5 is inclinedly wound around an outer side of the SUS rod 20 equal in diameter to the stent to form an inner artificial blood vessel layer 31, as shown in FIGS. 2A, 2B, 2C, 2D, 3A, 3B, 3C, and 3D.

The inner artificial blood vessel layer 31 is inserted and coupled with the stent 10, and another artificial blood vessel (PTFE; Polytetrafluoroethylene) 5 is inclinedly wound along an outer side of the stent 10, thus forming an outer artificial blood vessel layer 32.

For example, the inner artificial blood vessel layer 31 and the outer artificial blood vessel layer 32 are prepared to be positioned at the inside and outside, respectively, of the stent 10.

In this state, a silicone tube 40 is inserted into the SUS rod 20 to fasten the inner artificial blood vessel layer 31, the stent 10, and the outer artificial blood vessel layer 32.

In this case, the silicone tube 40 is about 2 mm to 3 mm larger in diameter than the SUS rod 20.

By doing so, the inner artificial blood vessel layer 31, the stent 10, the outer artificial blood vessel layer 32, and the silicone tube 40 are prepared to be sequentially positioned on the outside of the SUS rod 20.

The SUS rod 20 thusly prepared is fixedly mounted inside a vacuum heating device 50. Then, the inner artificial blood vessel layer 31 and the outer artificial blood vessel layer 32 positioned at the inside and outside of the hollow cylindrical body 11 of the stent 10 are attached by a vacuuming operation to each other towards the stent while internally and externally wrapping around the hollow cylindrical body 11, and the inner artificial blood vessel layer 31 and the outer artificial blood vessel layer 32 are thermally fused to each other by a heating operation, thereby forming a artificial blood vessel layer 30 in which the inner artificial blood vessel layer 31 and outer artificial blood vessel layer 32 are integrally attached to the stent 10.

In this case, fastening bolts may be adjusted in a screwing manner to pressurize and fasten both sides of the SUS rod 20. Preferably, one or more SUS rods 20 may be placed in the vacuum heating device 50 so that multiple stents may be processed at the same time.

Preferably, a heater (not shown) may be provided inside the SUS rod 20 so that the artificial blood vessel layers 31 and 32 may fused together by heat generated inside the SUS rod 20.

After the artificial blood vessel layer 30 is completely attached to the stent 10, the SUS rod 20 is separated from the vacuum heating device 50, and the silicone tube 40 and the stent 10 are sequentially separated from the SUS rod 20.

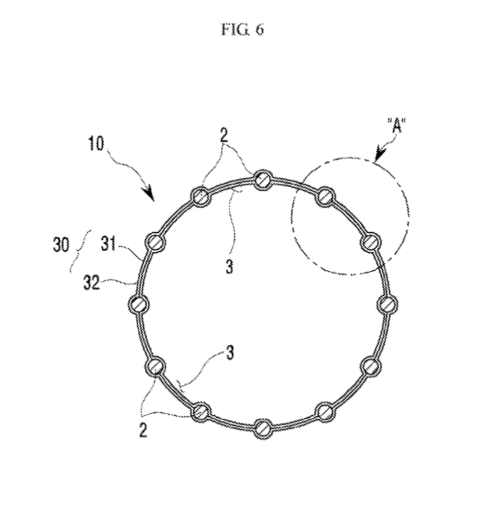

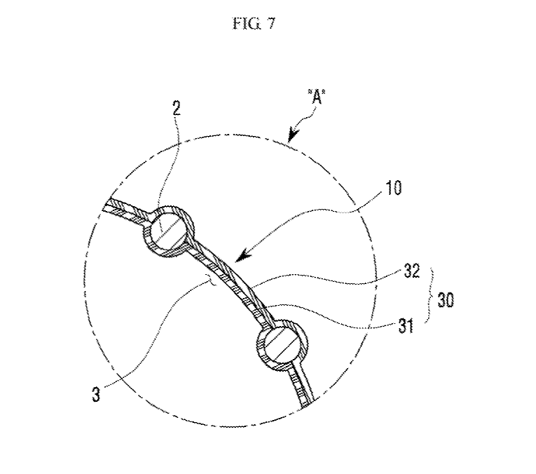

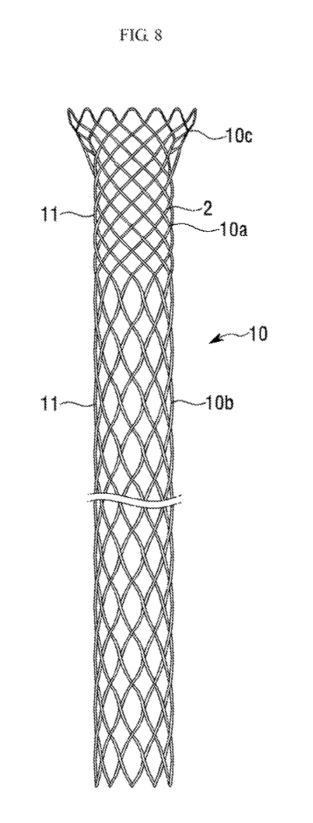

Both ends of the artificial blood vessel layer 30 of the stent 10 are finally cut to form an artificial blood vessel-attached stent 10 according to the present invention, as shown in FIGS. 5 to 7.

The inner artificial blood vessel layer 31 positioned inside the hollow cylindrical body 11 and the outer artificial blood vessel layer 32 positioned outside of the hollow cylindrical body 11 of the stent 10 are attached to each other while internally and externally wrapping around the hollow cylindrical body 11, and in this state, the inner artificial blood vessel layer 31 and the outer artificial blood vessel layer 32 are fused together by heat, thereby forming the artificial blood vessel layer 30 in which the inner artificial blood vessel layer 31 and outer artificial blood vessel layer 32 are fused together while integrally wrapping around the hollow cylindrical body 11 at the inside and outside thereof.

In other words, the inner artificial blood vessel layer 31 and the outer artificial blood vessel layer 32 respectively positioned at the inside and outside of the hollow cylindrical body 11 of the stent 10 internally and externally wrap around the superelastic shape memory alloy wires 2 and are fused together by heat in a space 3 between the superelastic shape memory alloy wires 2, with the inner artificial blood vessel layer 31 and the outer artificial blood vessel layer 32 attached to each other. Accordingly, unlike in the conventional art, the stent and the artificial blood vessel layer may be integrally attached to each other without any floating between the stent and the artificial blood vessel layer.

The artificial blood vessel-attached stent 10 according to the present invention may be inserted and placed in a lesion portion that is being stenosed or has been stenosed in a blood vessel or a lumen, such as the esophagus, duodendum, or colon, in the body, to expand the lesion portion, thereby securing a pathway.

In addition the above-described effects achieved by the artificial blood vessel layer 30 formed outside the hollow cylindrical body 11 of the stent 10, foods or other contents travelling through the pathway of a lumen in the body or blood in the vessel may be prevented from contacting the lesion portion, and the progress of the stenosis of the lesion portion may be prevented that may occur as the lesion portion grows toward the inside of the hollow cylindrical body 11 of the stent.

Further, unlike in the conventional art, the artificial blood vessel layer 30 formed at the hollow cylindrical body 11 of the stent 10 may be formed so that the inner artificial blood vessel layer 31 and the outer artificial blood vessel layer 32 respectively positioned at the inside and outside of the hollow cylindrical body 11 of the stent 10 internally and externally wrap around hollow cylindrical body 11 and are fused together by beat, with the inner artificial blood vessel layer 31 and the outer artificial blood vessel layer 32 attached to each other. Accordingly, unlike in the conventional art, the stent 10 and the artificial blood vessel layer 30 may be integrally attached to each other without any floating between the stent and the artificial blood vessel layer. Therefore, the above-described effects—e.g., foods or other contents travelling through the pathway of a lumen in the body or blood in the vessel may be prevented from contacting the lesion portion, and the progress of the stenosis of the lesion portion may be prevented that may occur as the lesion portion grows toward the inside of the hollow cylindrical body 11 of the stent—may be further enhanced.

In another embodiment, as shown in FIG. 8, one or more superelastic shape memory alloy wires 2 are woven to cross each other at a narrow interval, thus forming a dense stent 10a having a hollow cylindrical body.

The wires 2 are woven from an end of the dense stent 10a to cross each other at a broad interval, thus forming a sparse stent 10b having a hollow cylindrical body.

Further, the wires 2 are woven from another end of the dense stent 10a, forming a trumpet-shaped extension stent 10c. The extension stent 10c is coupled at the other end of the dense stent 10a, thus completing a stent 10.

The dense stent 10a and the sparse stent 10b are configured to have hollow cylindrical bodies 11, respectively, which have the same diameter.

In this case, the stent 10 is formed by a jig. To form the hollow cylindrical bodies 11 of the dense stent 10a and the sparse stent 10b, the jig forms a groove in a longitudinal direction at an equiangular position on the circumference of the cylindrical body, detachably couples pins to the groove at a predetermined distance, hangs the superelastic shape memory allow wires to the pins in a zig-zag pattern along the pins, and weaves the wires while bending and crossing the wires. The extension stent 10c is coupled with an end of the sparse stent 10b.

In particular, the hollow cylindrical body 11 of the stent may be formed to have various shapes and functions according to the patterns in which the superelastic shape memory alloy wires are hung to the jig pins in a zig-zag shape and woven, bent and crossed. According to the present invention, such stents having various shapes and functions all belong to the scope of the present invention.

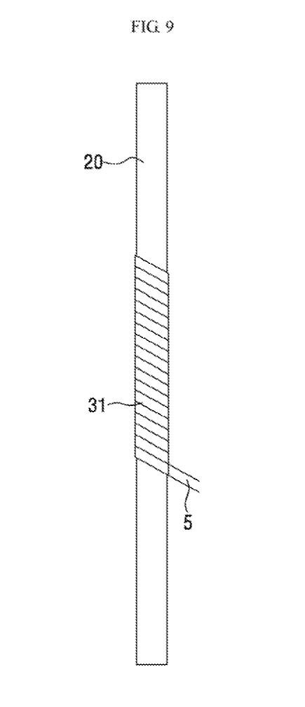

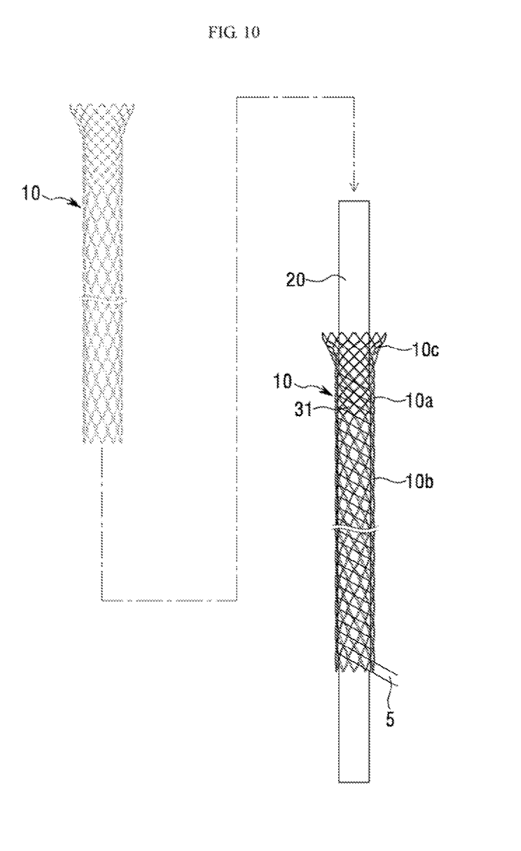

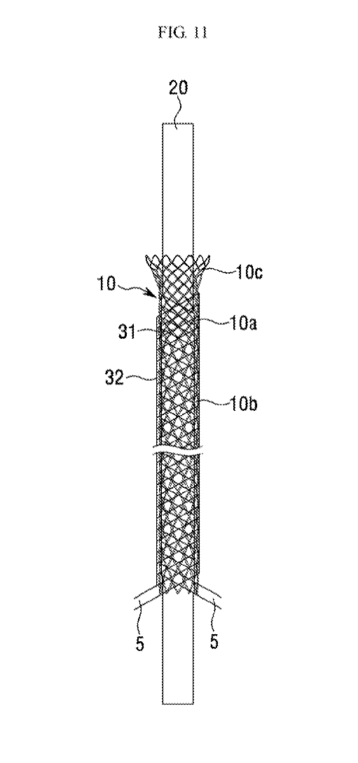

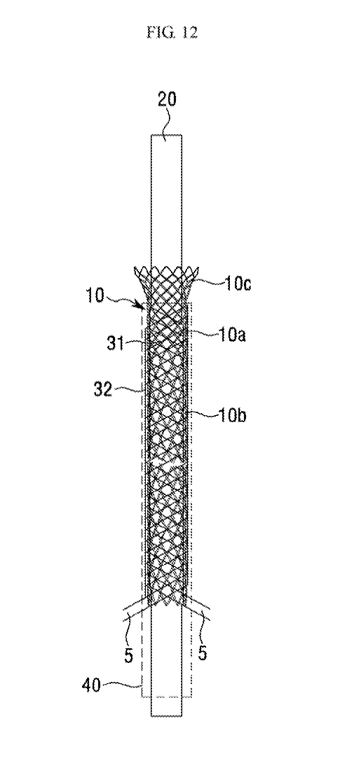

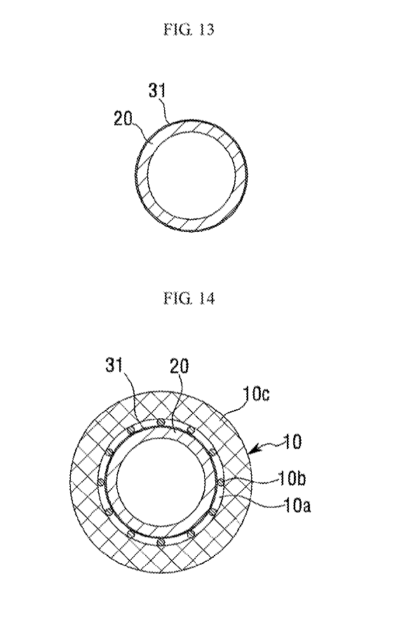

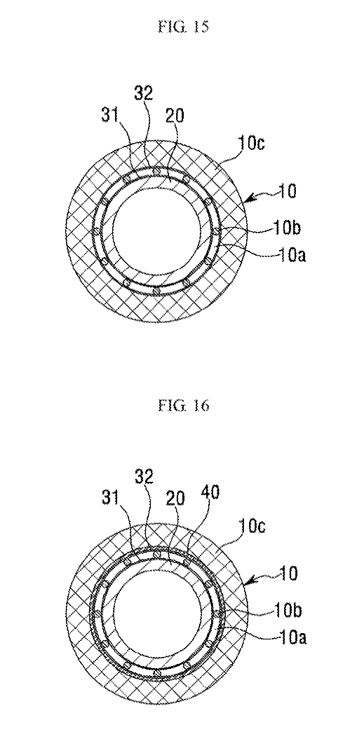

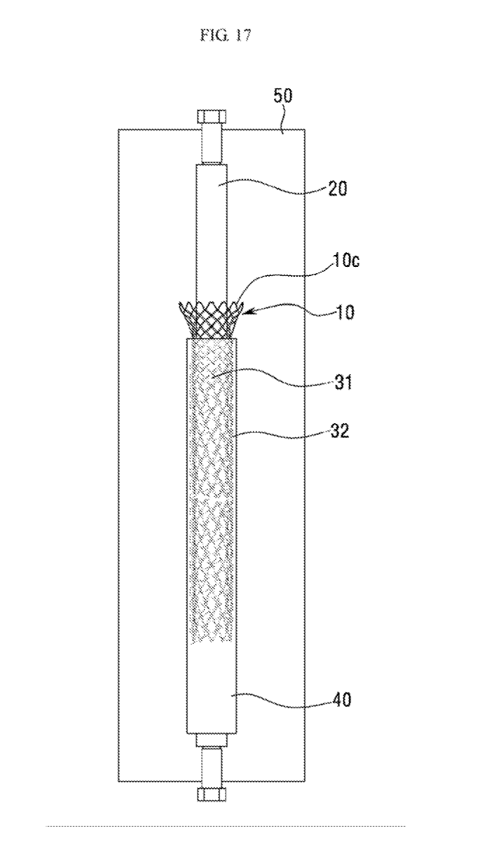

After forming the stent 10, an artificial blood vessel (PTFE; Polytetrafluoroethylene) 5 is inclinedly wound around an outer side of the SUS rod 20 equal in diameter to the stent to form an inner artificial blood vessel layer 31, as shown in FIGS. 9 to 17.

The inner artificial blood vessel layer 31 is inserted and coupled with the dense stent 10a and the sparse stent 10b of the stent 10, and another artificial blood vessel (PTFE; Polytetrafluoroethylene) 5 is inclinedly wound along an outer side of the dense stent 10a and the sparse stent 10b of the stent 10, thus forming an outer artificial blood vessel layer 32.

For example, the inner artificial blood vessel layer 31 and the outer artificial blood vessel layer 32 are prepared to be positioned at the inside and outside, respectively, of the dense stent 10a and the sparse stent 10b of the stent 10.

In this state, a silicone tube 40 is inserted into the SUS rod 20 to fasten the inner artificial blood vessel layer 31, the dense stent 10a and the sparse stent 10b of the stent 10, and the outer artificial blood vessel layer 32.

In this case, the silicone tube 40 is about 2 mm to 3 mm larger in diameter than the SUS rod 20.

By doing so, the inner artificial blood vessel layer 31, the dense stent 10a and the sparse stent 10b of the stent 10, the outer artificial blood vessel layer 32, and the silicone tube 40 are prepared to be sequentially positioned on the outside of the SUS rod 20.

The SUS rod 20 thusly prepared is fixedly mounted inside a vacuum heating device 50. Then, the inner artificial blood vessel layer 31 and the outer artificial blood vessel layer 32 positioned at the inside and outside of the hollow cylindrical bodies 11 of the dense stent 10a and the sparse stent 10b of the stent 10 are attached by a vacuuming operation to each other towards the stent while internally and externally wrapping around the hollow cylindrical bodies 11, and the inner artificial blood vessel layer 31 and the outer artificial blood vessel layer 32 are thermally fused to each other by a heating operation, thereby forming a artificial blood vessel layer 30 in which the inner artificial blood vessel layer 31 and outer artificial blood vessel layer 32 are integrally attached to the dense stent 10a and the sparse stent 10b.

In this case, fastening bolts may be adjusted in a screwing manner to pressurize and fasten both sides of the SUS rod 20. Preferably, one or more SUS rods 20 may be placed in the vacuum heating device 50 so that multiple stents 10 may be processed at the same time.

Preferably, a heater (not shown) may be provided inside the SUS rod 20 so that the artificial blood vessel layers 31 and 32 may fused together by heat generated inside the SUS rod 20.

After the artificial blood vessel layer 30 is completely attached to the dense stent 10a and the sparse stent 10b of the stent 10, the SUS rod 20 is separated from the vacuum heating device 50, and the silicone tube 40 and the stent 10 are sequentially separated from the SUS rod 20.

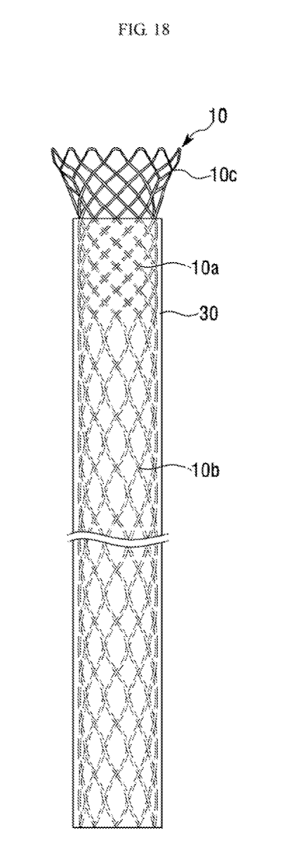

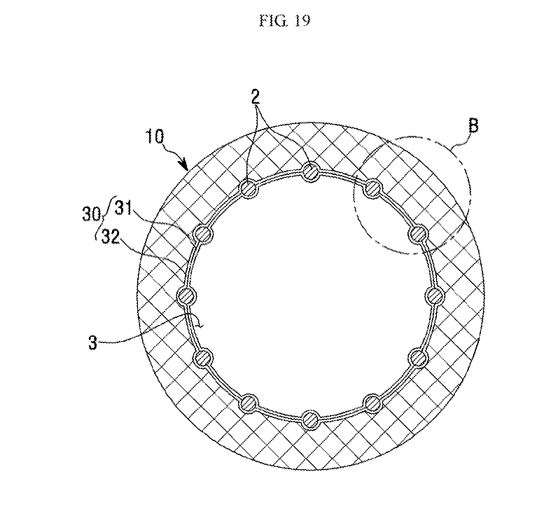

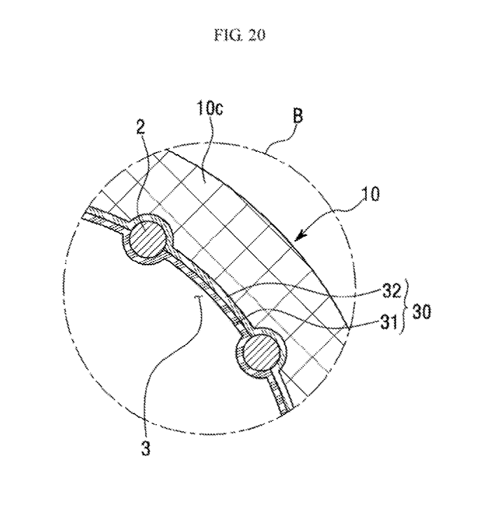

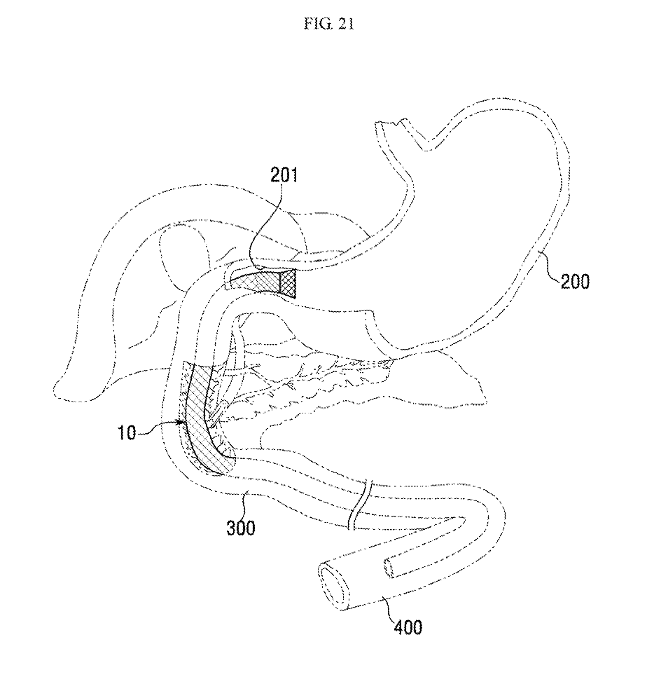

An end of the artificial blood vessel layer 30 of the stent 10 is finally cut to form an artificial blood vessel-attached stent 10 according to the present invention, as shown in FIGS. 18 to 20.

The inner artificial blood vessel layer 31 positioned inside the hollow cylindrical body 11 and the outer artificial blood vessel layer 32 positioned outside of the hollow cylindrical bodies 11 of the dense stent 10a and the sparse stent 10b of the stent 10 are attached to each other while internally and externally wrapping around the hollow cylindrical bodies 11, and in this state, the inner artificial blood vessel layer 31 and the outer artificial blood vessel layer 32 are fused together by heat, thereby forming the artificial blood vessel layer 30 in which the inner artificial blood vessel layer 31 and outer artificial blood vessel layer 32 are fused together while integrally wrapping around the hollow cylindrical bodies 11 at the inside and outside thereof.

In other words, the inner artificial blood vessel layer 31 and the outer artificial blood vessel layer 32 respectively positioned at the inside and outside of the hollow cylindrical body 11 of the stent 10 internally and externally wrap around the superelastic shape memory alloy wires 2 and are fused together by heat in a space 3 between the superelastic shape memory alloy wires 2, with the inner artificial blood vessel layer 31 and the outer artificial blood vessel layer 32 attached to each other. Accordingly, unlike in the conventional art, the stent and the artificial blood vessel layer may be integrally attached to each other without any floating between the stent and the artificial blood vessel layer.

The artificial blood vessel-attached stent 10 according to the present invention may be inserted and placed in a lesion portion that is being stenosed or has been stenosed in a blood vessel or a lumen, such as the esophagus, duodendum, or colon, in the body, to expand the lesion portion, thereby securing a pathway.

In addition the above-described effects achieved by the artificial blood vessel layer 30 formed outside the hollow cylindrical body 11 of the stent 10, foods or other contents travelling through the pathway of a lumen in the body or blood in the vessel may be prevented from contacting the lesion portion, and the progress of the stenosis of the lesion portion may be prevented that may occur as the lesion portion grows toward the inside of the hollow cylindrical body 11 of the stent.

Further, unlike in the conventional art, the artificial blood vessel layer 30 formed at the hollow cylindrical body 11 of the stent 10 may be formed so that the inner artificial blood vessel layer 31 and the outer artificial blood vessel layer 32 respectively positioned at the inside and outside of the hollow cylindrical body 11 of the stent 10 internally and externally wrap around hollow cylindrical body 11 and are fused together by beat, with the inner artificial blood vessel layer 31 and the outer artificial blood vessel layer 32 attached to each other. Accordingly, unlike in the conventional art, the stent 10 and the artificial blood vessel layer 30 may be integrally attached to each other without any floating between the stent and the artificial blood vessel layer. Therefore, the above-described effects—e.g., foods or other contents travelling through the pathway of a lumen in the body or blood in the vessel may be prevented from contacting the lesion portion, and the progress of the stenosis of the lesion portion may be prevented that may occur as the lesion portion grows toward the inside of the hollow cylindrical body 11 of the stent—may be further enhanced.

As shown in FIG. 21, the stent 10 including the dense stent 10a, the sparse stent 10b, and the extension stent 10c, when applied to an obese patient, passes through the inside of his stomach so that the extension stent 10c is stuck in the pylorus where the stomach 200 and the duodenum 300 are connected and so that the dense stent 10a and the sparse stent 10b are inserted to an entrance of the small intestine 400 via the duodenum 300.

For example, the dense stent 10a and the sparse stent 10b prevents the food in the stomach 200 from traveling thus preventing the food from being mixed with bile secreted from the pancreas to the duodenum 300. Thus, digestion may be obstructed to reduce absorption of nutrients in the duodenum 300, and the foods aiding in decreasing obesity may be directly conveyed to the small intestine 400.

In this case, the extension stent 10c is stuck in the pylorus 201 of the stomach 200, hampering the movement of the stent 10. The dense stent 10a, although positioned at the pylorus 201 being contracted, is not easily deformed thanks to being densely woven and remains at its shape in a straight line portion connected to the duodenum 300.

Further, the sparse stent 10b connected with the dense stent 10a is inserted to the small intestine 400 via the duodenum 300. Since the sparse stent 10b is sparsely woven at a broad interval between the wires, the sparse stent 10b may be easily bent in the duodenum 300 or small intestine 400 with many bends or curves.

What is claimed is:

1. A method of manufacturing an artificial blood vessel-attached stent, the method comprising:
forming a stent having a hollow cylindrical body by crossingly weaving a superelastic shape memory alloy wire;
performing attachment preparation by forming an inner artificial blood vessel layer by inclinedly winding an artificial blood vessel (PTFE: Polytetrafluoroethylene) along an outer side of a SUS rod to form an inner artificial blood vessel layer, inserting the inner artificial blood vessel layer into the stent, inclinedly winding an artificial blood vessel (PTFE; Polytetrafluoroethylene) along an outer side of the stent to form an outer artificial blood vessel layer, and inserting a silicone tube; and
fixedly mounting the SUS rod inside a vacuum heating device, attaching the inner artificial blood vessel layer and the outer artificial blood vessel layer positioned at the inside and outside of the stent to each other by a vacuuming operation towards the stent with the inner artificial blood vessel layer and the outer artificial blood vessel layer internally and externally wrapping around the stent, and thermally fusing the inner artificial blood vessel layer and the outer artificial blood vessel layer to each other by a heating operation so that the inner artificial blood vessel layer and outer artificial blood vessel layer are integrally attached to the stent.

2. The method of claim 1, wherein the heating operation of the vacuum heating device is performed by a heater provided inside the SUS rod.

3. A artificial blood vessel-attached stent comprising an artificial blood vessel layer at an outside of a hollow cylindrical body formed by crossingly weaving a superelastic shape memory alloy wire, wherein the artificial blood vessel layer is formed by thermally fusing an inner artificial blood vessel layer and an outer artificial blood vessel layer respectively positioned at an inside and outside of the hollow cylindrical body of the stent, with the inner artificial blood vessel layer and the outer artificial blood vessel layer attached with each other while internally and externally wrapping around the hollow cylindrical body.

4. A method of manufacturing an artificial blood vessel-attached stent, the method comprising:
forming a stent by forming a dense stent having a hollow cylindrical body by crossingly weaving a superelastic shape memory alloy wire to have a narrow interval, forming a sparse stent having a hollow cylindrical body by crossingly weaving the superelastic shape memory alloy wire from an end of the dense stent to have a broad interval, and forming an extension stent having a trumpet-shaped body to weaving the superelastic shape memory alloy wire from another end of the dense stent and coupling the extension stent to the other end of the dense stent;
performing attachment preparation by forming an inner artificial blood vessel layer by inclinedly winding an artificial blood vessel (PTFE; Polytetrafluoroethylene) along an outer side of a SUS rod to form an inner artificial blood vessel layer, inserting the inner artificial blood vessel layer into the dense stent and the sparse stent, inclinedly winding an artificial blood vessel (PTFE; Polytetrafluoroethylene) along an outer side of the dense stent and the sparse stent to form an outer artificial blood vessel layer, and inserting a silicone tube; and
fixedly mounting the SUS rod inside a vacuum heating device, attaching the inner artificial blood vessel layer and the outer artificial blood vessel layer positioned at the inside and outside of the dense stent and the sparse stent to each other by a vacuuming operation towards the dense stent and the sparse stent with the inner artificial blood vessel layer and the outer artificial blood vessel layer internally and externally wrapping around the dense stent and the sparse stent, and thermally fusing the inner artificial blood vessel layer and the outer artificial blood vessel layer to each other by a heating operation so that the inner artificial blood vessel layer and outer artificial blood vessel layer are integrally attached to the dense stent and the sparse stent.

5. The method of claim 4, wherein the heating operation of the vacuum heating device is performed by a heater provided inside the SUS rod.

6. A artificial blood vessel-attached stent comprising a hollow cylindrical body including a dense stent formed by weaving a superelastic shape memory alloy wire at a narrow interval in a portion thereof, a sparse stent formed by weaving the superelastic shape memory alloy wire from a side of the dense stent at a broad interval and a trumpet-shaped extension stent coupled with an end of the dense stent, wherein an artificial blood vessel layer is formed at an outside of the hollow cylindrical body, and wherein the artificial blood vessel layer is formed by thermally fusing an inner artificial blood vessel layer and an outer artificial blood vessel layer respectively positioned at an inside and outside of the hollow cylindrical body of the stent, with the inner artificial blood vessel layer and the outer artificial blood vessel layer attached with each other while internally and externally wrapping around the hollow cylindrical body.
